(19) United States
(12) Patent Application Publication
(10) Pub. No.: US 2025/0073281 A1
(43) Pub. Date: Mar. 6, 2025

(54) LACTOBACILLUS REUTERI HAVING IMPROVED ANTI-AGING AND HAIR HEALTH ENHANCEMENT EFFECTS, AND APPLICATION THEREOF

(71) Applicants: QINGDAO VLAND BIOTECH INC., Qingdao, Shandong (CN); QINGDAO VLAND BIOTECH GROUP CO., LTD., Qingdao, Shandong (CN)

(72) Inventors: Zhi DUAN, Qingdao, Shandong (CN); Luxi ZHANG, Qingdao, Shandong (CN); Hongchang CUI, Qingdao, Shandong (CN); Jingyan ZHANG, Qingdao, Shandong (CN); Chaoqun GUO, Qingdao, Shandong (CN)

(73) Assignees: QINGDAO VLAND BIOTECH INC., Qingdao, Shandong (CN); QINGDAO VLAND BIOTECH GROUP CO., LTD., Qingdao, Shandong (CN)

(21) Appl. No.: 18/555,869

(22) PCT Filed: Jun. 21, 2021
(86) PCT No.: PCT/CN2021/101274

§ 371 Date: Oct. 18, 2023

(30) Foreign Application Priority Data
Apr. 18, 2021 (CN) 202110415247.X

(51) Int. Cl. A61K 35/742; A61P 17/00; C12N 1/20; C12R 1/225

(52) U.S. Cl. CPC A61K 35/742; A61P 17/00; C12N 1/205; C12R 2001/225

(57) ABSTRACT

Provided is a novel strain of Lactobacillus reuteri, which is preserved in the China Center for Type Culture Collection on 25 Jan. 2021 as Lactobacillus reuteri VHPribo E18 having a preservation number of CCTCC NO: M2021153. The provided Lactobacillus reuteri VHProbi E18 has strong tolerance for artificial intestine gastric fluids and can be germinated in the artificial intestine gastric fluids. The strain is sensitive to common antibiotics such as erythromycin and ampicillin, does not produce hemolysin, cannot dissolve blood cells, and has excellent biological safety. The provided Lactobacillus reuteri VHPribo E18 can be used for preparing functional food or cosmetics which have the effects of delaying and repairing skin aging, and has wide application prospects.

This application claims the priority of Chinese Patent Application No. 202110415247.X, filed with the China National Intellectual Property Administration on Apr. 18, 2021, and titled with “LACTOBACILLUS REUTERI HAVING IMPROVED ANTI-AGING AND HAIR HEALTH ENHANCEMENT EFFECTS, AND APPLICATION THEREOF”, which is hereby incorporated by reference in entirety.

### FIELD

The present invention belongs to the technical field of probiotic screening and application, and specifically relates to a strain of Lactobacillus reuteri having effects of ameliorating aging skin and enhancing hair health and use thereof.

### BACKGROUND

China is becoming more of an aging society, the elderly population accounts for an increasing proportion of the overall population. A range of health issues associated with human aging, such as skin sagging and hair loss, have gradually arisen and troubled people's daily lives, impacting their quality of life. Existing drugs for improving skin health and preventing or treating hair loss not only require long-term use, but also have serious side effects. The research and development of anti-aging active substances has become a hotspot in response to the aging population.

Studies have found that probiotics play an important role in delaying human aging. Increasing evidence demonstrates that the influence of probiotics on human health is not restricted to the intestinal tract, but extends to wider effects, including regulation of endocrine balance, immune balance, nervous system and respiratory system. Therefore, taking probiotics to delay aging and improve skin health has become a new treatment idea.

### SUMMARY

A purpose of the present invention is to provide a novel strain of Lactobacillus reuteri and use thereof. The provided strain of Lactobacillus reuteri, isolated from the fresh feces of a centenarian, can increase the collagen content of the skin, ameliorate skin sagging, improve the health of hair follicles, protect hair, ameliorate skin aging, and have anti-aging effects.

The strain of Lactobacillus reuteri provided by the present invention is Lactobacillus reuteri VHPribo E18 strain, which has been deposited in the China Center for Type Culture Collection on Jan. 25, 2021, with the deposit number of CCTCC NO: M2021153.

The Lactobacillus reuteri VHPribo E18 strain provided by the present invention has a fingerprint pattern by Riboprinter as shown in FIG. 2, a fingerprint pattern of RAPD as shown in FIG. 3, and a fingerprint pattern of rep-PCR as shown in FIG. 4.

The Lactobacillus reuteri VHPribo E18 strain provided by the present invention is used for the preparation of a product with antioxidant function.

The Lactobacillus reuteri VHPribo E18 strain provided by the present invention can also be used for the preparation of a product with the effect of delaying and repairing skin aging.

The product is preferably a food supplement. The product is preferably a cosmetic.

The Lactobacillus reuteri VHProbi E18 strain provided by the present invention has strong tolerance to artificial gastrointestinal fluid and can germinate in artificial gastrointestinal fluid. The strain is sensitive to common antibiotics such as erythromycin and ampicillin, and does not produce hemolysin or lyse blood cells, indicating good biological safety.

The strain exhibits significant antioxidant activity. The bacteria display an anti-lipid peroxidation inhibition rate of 6.76%, the supernatant exhibits a rate of 9.93%, and the intracellular extract shows a rate of 10.68%. Furthermore, the DPPH clearance rate amounts to 32.8% and the HRS clearance rate amounts to 4.32%. The strain can also degrade cholesterol, with a degradation rate of 16.24%. Moreover, the strain's cell surface hydrophobicity is 61.7%, and its adhesion capacity is 4.7.

In research on anti-aging efficacy in skin, compared with the mice in the 12-month-old control group, the glossiness of the fur of the mice in the 12-month-old gavage group and the 12-month-old smearing group was improved, with no white hair and less hair loss. For the mice in the 12-month-old gavage group and the 12-month-old smearing group, the skin moisture content increased, the MDA content decreased, and the content of hydroxyproline in the skin and tail tendon increased, indicating that smearing and gavage of the Lactobacillus reuteri VHProbi E18 strain can increase the content of collagen in the dermis. According to the histological examination of mouse skin, compared with the mice in the 12-month-old control group, the mice in the 12-month-old gavage group and the 12-month-old smearing group had an improved integrity of the epidermal structure, reduced inflammatory cells, and increased fibroblasts, and the effect of the gavage group was better than that of the smearing group. In addition, the number of skin hair follicles, dermis thickness, and collagen fiber content of the mice in the 12-month-old gavage group and 12-month-old smearing group increased. The results show that the Lactobacillus reuteri VHProbi E18 strain can improve the skin signs of aging mice and achieve the effect of delaying and repairing skin aging, whether administered by gavage or smearing.

The Lactobacillus reuteri VHProbi E18 strain provided by the present invention has no toxic effect on the body. It can be added to food to prepare food supplement with anti-aging effect, and can also be added to cosmetics to delay skin aging, showing broad application prospects.

### BRIEF DESCRIPTION OF DRAWINGS

FIG. 1 shows the carbon source metabolism map of E18 strain by API 50CHL.

FIG. 2 shows the fingerprint pattern of strain E18 by Riboprinter.

FIG. 3 shows the RAPD fingerprint of E18 strain.

FIG. 4 shows the rep-PCR fingerprint of E18 strain.

FIG. 5 shows the sensory score of mouse fur in each group.

FIG. 6 shows the determination results of skin moisture content of mice in each group.

FIG. 7 shows the determination results of MDA content in the skin of mice in each group.

FIG. 8 shows the determination results of hydroxyproline content in the skin of mice in each group.

FIG. 9 shows the results of HE staining of mouse skin in each group, including (A) 3-month-old control group; (B) 12-month-old control group; (C) 12-month-old smearing group; and (D) 12-month-old gavage group.

FIG. 10 shows the determination results of the number of skin hair follicles, epidermal thickness, dermal thickness and collagen fiber content of mice in each group, including (A) 3-month-old control group; (B) 12-month-old control group; (C) 12-month-old smearing group; and (D) 12-month-old gavage group. *: compared with 3-month-old control group, p<0.05; **: compared with 12-month-old control group, p<0.05.

### DETAILED DESCRIPTION

The Lactobacillus reuteri VHProbi E18 strain provided by the present invention meets regulatory requirements and can be used as a source of food raw materials. Long-term use will not cause side effects or the risk of overdose. After polyphasic taxonomic identification, the Lactobacillus reuteri VPHrobi E18 strain is a novel strain. The Lactobacillus reuteri VHProbi E18 strain provided by the present invention has anti-aging effects, which can ameliorate age-related aging problems such as skin aging when used alone without compounding with prebiotics and/or other probiotics, and has important application value.

The applicant deposited the Lactobacillus reuteri VHProbi E18 strain in the China Center for Type Culture Collection of Wuhan University on Jan. 25, 2021, with the deposit number of CCTCC NO: M2021153.

The screening method of the present invention is not limited to the examples, and known methods that can achieve the purpose of screening can be used. The description of screening in the examples is only illustrative of the present invention and does not limit the scope of protection of the present invention. Without departing from the spirit and essence of the present invention, any modifications or substitutions made to the method, steps or conditions of the present invention shall fall within the scope of the present invention.

The present invention will be described in detail below in conjunction with specific examples.

### Example 1 Isolation and Screening of Lactobacillus reuteri VHProbi E18

### 1. Preliminary Screening

MRS agar medium was prepared, adjusted to pH 6.2-6.5, and sterilized under high pressure at 121° C. for 15 min.

1 g of fresh fecal samples from a centenarian (the sampling process complied with the ethical standards of biological sampling) was diluted with sterile saline, placed into a sterile sample bag, beat and mixed well with a homogenizer. 100 uL of the mixture was diluted in gradient, spread on MRS agar medium and incubated anaerobically at 37° C. for 48 h. When a single colony grew on the plate, microscopic examination was performed. According to the microscopic examination results, the applicant screened a total of 20 potential lacti acid bacterial strains, which were named E01, E02, . . . , E18, E19, and E20.

### 2. Re-Screening

1 L of MRS liquid culture medium was prepared and sterilized under high pressure at 121° C. for 15 min. After the culture medium cooled, 3.2 g of porcine mucosal pepsin was added, dissolved under shaking, and placed in a 37° C. water bath shaker for 1 h to prepare an acid-resistant culture medium.

The strains E01, E02, . . . , E18, E19, and E20 obtained by screening were inoculated into

the above-mentioned acid-resistant medium at an inoculation amount of 6%, and cultured anaerobically and statically for 48 h at 37° C. The fermentation broth was then taken for bacteria count.

The results showed that among the log values of the viable bacteria in the fermentation broth of the 20 Lactobacillus strains, the E18 strain had the largest number of viable bacteria after re-screening on the acid-resistant medium, with a log value as high as 8.01 Log CFU/mL, indicating that the E18 strain had the highest acid resistance.

### Example 2 Identification of Strains

### 1. Identification of Colony Morphology

The E18 strain was inoculated on the MRS agar medium. After anaerobic culture at 37° C. for 24 h, E18 single colony was milky white, with a colony diameter of about 1.5-2 mm and a smooth surface, presenting as campylobacter with rounded ends under microscope.

### 2. Identification of Physiological and Biochemical Characteristics

The preparation of the inoculum in this example was performed as follows: under sterile conditions, an appropriate amount of fresh E18 strain was centrifuged at 5000 rpm/min for 5 min, washed twice with PBS buffer, resuspended in PBS buffer with the same volume, and then diluted 50 times to obtain the inoculum.

### 2.1. Salinity Tolerance Test

Under sterile conditions, 190 μL of BSM liquid culture medium with salt concentrations of 1%, 2%, 3%, 4%, 5%, 6%, 7%, and 8% was added to the 96-well plate, respectively, with 3replicates for each salt concentration. Then 10 μL of the inoculum was added, and the wells without bacteria were used as controls. 50 μL of paraffin oil sterilized under high pressure was added to each well to prevent water evaporation during culture. The plate was then placed at 37° C. for incubation at constant temperature, and turbidity of the culture medium was observed. The results showed that tolerance to the maximum salt concentration of E18 strain was 5%.

### 2.2. Catalase Experiment

A drop of the fresh bacterial liquid was dripped on a clean glass slide, and then added with a drop of 3% hydrogen peroxide solution. It was observed that the E18 strain did not produce bubbles, showing a negative reaction.

### 2.3. Carbon Source Metabolism Test

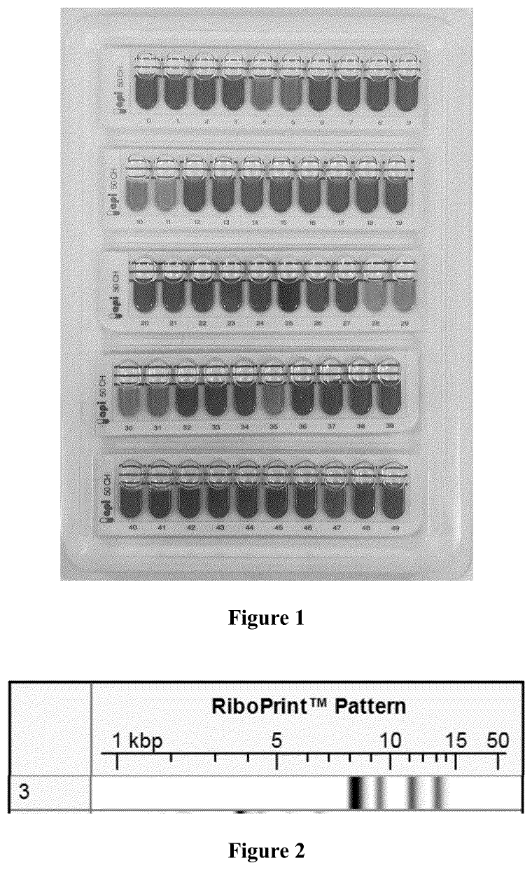

The carbon source metabolism test was performed on E18 strain using API 50CHL reagent strips. For details on experimental methods and result interpretation, please refer to the API 50CHL kit instructions. The identification result of E18 strain was: % ID=93 and T value=0.74, API result showing Lactobacillus fermentum with a good identification based on the identification comment (there was currently no Lactobacillus reuteri among the species that can be identified by API, so the identification result was Lactobacillus fermentum). The results of carbon source metabolism of E18 strain by API 50CHL are shown in FIG. 1.

### 3. Molecular Biology Identification

3.1 16s rDNA gene sequence analysis
3.1.1. Genomic DNA extraction

The operation was carried out according to the Tiangen Bacterial Genomic DNA Extraction Ki (Cat. No.: DP302).

3.1.2. 16s rDNA gene amplification

Primer sequence:

27F:
AGAGTTTGATCCTGGCTCA;
1492R:
GGTTACCTTGTTACGACTT.

The 16s rDNA sequence of the E18 strain was obtained by sequencing, as shown in SEQ ID NO: 1. The sequence was blasted in the NCBI database, and the E18 strain was initially determined as Lactobacillus reuteri.

### 3.2 Riboprinter Fingerprint Pattern

The purified single colony was picked up from the agar medium plate by a sampling stick, and placed into a sample tube containing buffer. A hand mixer was used for stirring to suspend the bacteria in the buffer, and then the sample rack was placed into a heater for inactivation and then put into a Riboprinter system. After the sample underwent DNA preparation, membrane transfer, imaging detection and data processing, the bacterial identification results were obtained. The identification results showed that the E18 strain was Lactobacillus reuteri, and its Riboprinter fingerprint results are shown in FIG. 2.

3.3 RAPD and rep-PCR fingerprint pattern identification
3.3. 1. RAPD fingerprint pattern identification

Primer sequence: GAGGGTGGCGGTTCT.

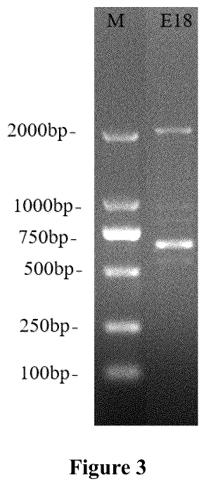

The RAPD fingerprint pattern of E18 strain is shown in FIG. 3.

3.3.2. rep-PCR fingerprint pattern

Primer sequence: CTACGGCAAGGCGACGCTGACG.

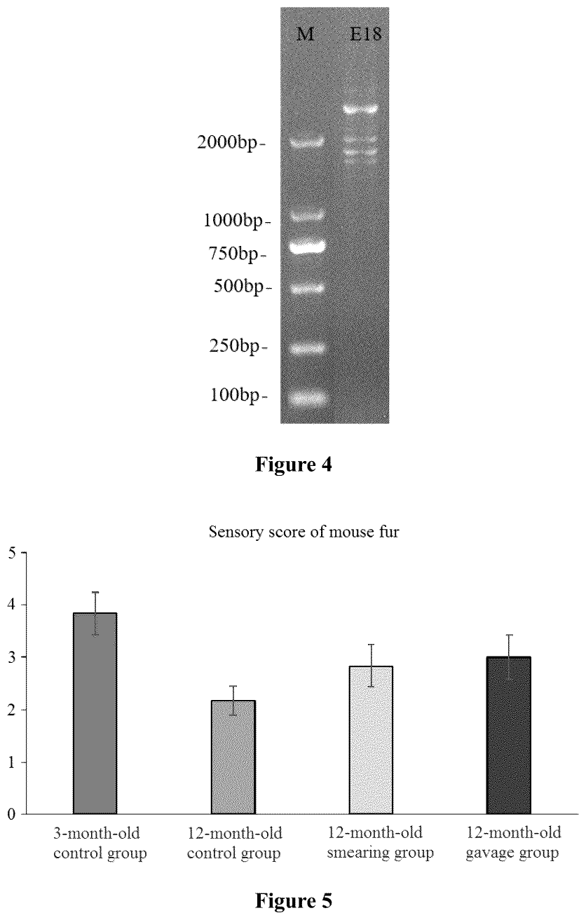

The rep-PCR fingerprint pattern of E18 strain is shown in FIG. 4.

The colony morphology and physiological and biochemical characteristics of the E18 strain were uploaded to the website, http://www.tgw1916.net/bacteria_logare_desktop.htmL, and combined with results published in the literature De Clerck E, et al. Systematic and applied microbiology, 2004, 27(1)50 for comparison. Based on the identification results of molecular biology, the E18 strain was determined to be a new strain of Lactobacillus reuteri and named Lactobacillus reuteri VHProbi E18.

### Example 3 Tolerance Test of Lactobacillus reuteri VHProbi E18 to Artificial Gastric Fluid and Artificial Intestinal Fluid

### 1. Preparation of Artificial Gastric Fluid

5 g of peptone, 2.5 g of yeast extract, 1 g of glucose and 2 g of NaCl were added into 1000 mL of distilled water, adjusted to pH 3.0 with dilute hydrochloric acid, and then sterilized at 115° C. for 20 min. Then 3.2 g of porcine mucosal pepsin was added before use, dissolved under shaking, and placed in a warm water bath at 37° C. in a water bath shaker for 1 h to simulate human body temperature.

### 2. Preparation of Artificial Intestinal Fluid

5 g of peptone, 2.5 g of yeast extract, 1 g of glucose, 6.8 g of KH₂PO₄ and 3.0 g of ox bile salt were added into 77 mL of 0.2 mol/L NaOH solution, adjusted to volume of 1000 mL, adjusted to pH 6.8±0.1 with dilute hydrochloric acid or sodium hydroxide solution, and sterilized at 115° C. for 20 min. Then 1 g of trypsin was added before use, dissolved under shaking, and placed in a 37° C. water bath shaker for 1 h to simulate human body temperature.

### 3. Test Method

2 mL of fresh bacterial liquid was centrifuged at 5000 rpm/min for 5 min to collect the bacteria, which was then washed 3 times with physiological saline, and then resuspended in 2 mL of physiological saline to serve as the inoculum. 1 mL of the inoculum was added to 24 mL of artificial intestinal fluid, and placed in a 37° C. water bath shaker (200 rpm/min) for 3 h. 1 mL of the sample was taken to detect the amount of viable bacteria.

The viable bacterial count was determined in accordance with the national standard “GB4789.35-2016-Food Microbiological Testing of Lactic Acid Bacteria”. The viable bacterial count (Log CFU/mL) of the strain after digestion by artificial intestinal fluid is shown in Table 1.

TABLE 1
Viable bacterial count after digestion
by artificial gastrointestinal fluid

| | After digestion by | After digestion by |
|---|---|---|
| Before digestion | artificial gastric fluid | artificial intestinal fluid |
| 7.97 ± 0.05 | 8.10 ± 0.06 | 7.98 ± 0.02 |

It can be seen from Table 1 that after Lactobacillus reuteri VHProbi E18 screened in the present invention was digested by artificial gastric fluid and artificial intestinal fluid, the amount of viable bacteria increased. This shows that the strain can tolerate artificial gastric fluid and artificial intestinal fluid, and can further undergo certain germination.

### Example 4 Hemolysis and Antibiotic Tolerance Experiments of Lactobacillus reuteri VHProbi E18

### 1. Hemolysis Experiment

Each component of the TBS basic culture medium was weighed, dissolved, and sterilized under high pressure at 121° C. for 15 min. When the culture medium cooled to 50° C., 5% sterile defibrinated sheep blood was added, mixed well, and poured into a plate. The test strain was inoculated on the prepared blood cell plate by streaking, and cultured in a 37° C. incubator. After 24 to 48 h, the hemolysis of test strain had was observed.

The results showed that Lactobacillus reuteri VHProbi E18 could not grow and there was no change in the blood cell plate, indicating that Lactobacillus reuteri VHProbi E18 did not produce hemolysin and was unable to lyse blood cells.

### 2. Antibiotic Tolerance Experiment

The minimum inhibitory concentration (MIC) value of antibiotics against Lactobacillus reuteri VHProbi E18 was determined by the broth microdilution method. The specific results are shown in Table 2.

TABLE 2
MIC values of antibiotics against Lactobacillus reuteri VHProbi E18

| Erythromycin | | Gentamicin | | Streptomycin | | Ampicillin | | Tetracycline | | Clindamycin | |
|---|---|---|---|---|---|---|---|---|---|---|---|
| MIC | R/S | MIC | R/S | MIC | R/S | MIC | R/S | MIC | R/S | MIC | R/S |
| 1 | / | 256 | 1 | 512 | / | 1 | / | 16 | / | 256 | / |

MIC unit: μg/mL

It can be seen from the results in Table 2 that Lactobacillus reuteri VHProbi E18 provided by the present invention was sensitive to common antibiotics such as erythromycin and ampicillin, and had good biological safety.

### Example 5 Determination of antioxidant function of Lactobacillus reuteri VHProbi E18

1. Determination of DPPH (1, 1-diphenyl-2-trinitrophenylhydrazine) Scavenging and Hydroxyl Radical Scavenging (HRS) Capacity of the Strain

Determination of DPPH (1, 1-diphenyl-2-trinitrophenylhydrazine) radical scavenging capacity of the strain

1 mL of the bacterial suspension of the strain to be tested in PBS was added with 1 mL of 0.4 mM freshly prepared DPPH free radical solution, mixed evenly, and then placed at room temperature for 30 min in the dark. Then the absorbance of the sample at a wavelength of 517 nm was measured as Aₛₐₘₚₗₑ for 3 replicates. The samples in the control group were zeroed using equal volumes of PBS solution and a mixture of DPPH and ethanol, and equal volumes of bacterial suspension in PBS and ethanol mixture. The scavenging rate was calculated according to the following formula: scavenging rate %=[1−(Aₛₐₘₚₗₑ−A_{blank})/A_{control}]×100%. The results are shown in Table 3.

TABLE 3
DPPH radical scavenging rate

| Strain | Scavenging rate % | Standard deviation |
|---|---|---|
| Lactobacillus reuteri | 32.88% | 2.51% |
| VHProbi E18 | | |

3) Determination of hydroxyl radical scavenging (HRS) capacity of the strain

100 μL of 5 mM sodium salicylate-ethanol solution, 100 μL of 5 mM ferrous sulfate, 500 μL of deionized water and 200 μL of lactic acid bacteria suspension in PBS were mixed well, and 100 μL of hydrogen peroxide solution (3 mM) was added. After 15 min in a 37° C. water bath, the absorbance of the sample was measured at a wavelength of 510 nm. The hydroxyl radical scavenging rate was calculated according to the following formula.

Scavenging rate %=(Aₛₐₘₚₗₑ−A_{control})/(A_{blank}−A_{control})×100%, where A_{control} is a deionized water replacement sample, and A_{blank} is a deionized water replacement sample and H₂O₂. The results are shown in Table 4.

TABLE 4
Hydroxyl radical scavenging (HRS) rate

| Strain | Scavenging rate % | Standard deviation |
|---|---|---|
| Lactobacillus reuteri | 4.32% | 1.06% |
| VHProbi E18 | | |

### 2. Experimental Identification of Anti-Lipid Peroxidation of Strain

Preparation of linoleic acid emulsion: 0.1 mL linoleic acid, 0.2 mL Tween 20, 19.7 mL deionized water

To 0.5 mL of PBS solution (pH 7.4), 1 mL of linoleic acid emulsion, 1 mL of FeSO₄ (1%) and 0.5 mL of sample were added. Then the mixture was placed in a 37° C. water bath for 1.5 h, then added with 0.2 mL of TCA (4%) and 2 mL of TBA (0.8%), subjected to 100° C. water bath for 30 min, cooled quickly and centrifuged at 4000 rpm/min for 15 min. The supernatant was collected to measure the absorbance at 532 nm, expressed as A. The control group used 0.5 mL of distilled water instead of the sample, and the absorbance was expressed as A₀. Inhibition rate/%=(A0−A)/A₀×100%

Note: A represents the absorbance of the sample group; and A0 represents the absorbance of the control group. The results are shown in Table 5.

TABLE 5
Anti-lipid peroxidation inhibition rate

| | Inhibition rate | Standard deviation |
|---|---|---|
| Bacteria | 6.76% | 1.41% |
| Fermentation supernatant | 9.93% | 0.80% |
| Intracellular extract | 10.68% | 1.75% |

### Example 6 In vitro Cholesterol Degradation Experiment of Lactobacillus reuteri VHProbi E18

1. Preparation of cholesterol micelle solution: 1 g of cholesterol was accurately weighed, dissolved in absolute ethanol, adjusted to volume of 100 mL, and filtered with a 0.22 μm microporous filter membrane under sterile conditions.

2. 10.0 g of peptone, 10.0 g of beef extract, 5.0 g of yeast extract, 2.0 g of diammonium hydrogen citrate, 20.0 g of glucose, 1.0 mL of Tween 80, 5.0 g of sodium acetate, 0.1 g of magnesium sulfate, 0.05 g of manganese sulfate, 2.0 g of dipotassium hydrogen phosphate, and 1g of bile salt were added into 1000 mL of distilled water, adjusted to pH 7.3, sterilized at 115° C. for 30 min, and then added with cholesterol solution to make the final concentration of cholesterol 0.1%.

Fresh bacterial liquid was inoculated at an inoculum amount of 0.1%, and incubated statically at 37° C. for 48 h. Then 0.2 mL of bacterial liquid was added with 1.8 mL of absolute ethanol, mixed, left for 10 min, and centrifuged at 3000 rpm for 5 min to obtain the supernatant for determination of cholesterol content. The cholesterol determination was performed in accordance with GB/T 5009.128-2003<Determination of Cholesterol in Foods>.

The results show that the degradation rate of cholesterol by Lactobacillus reuteri VHProbi E18 provided by the present invention reached 16.24% (bile salt-free data).

### Example 7 Cell Surface Hydrophobicity Test of Lactobacillus reuteri VHProbi E18

1. Preparation of bacterial liquid to be tested: The purified Lactobacillus reuteri VHProbi E18 colony was picked and inoculated into a newly prepared MRS liquid medium, cultured at 37° C. for 24 to 48 h, then inoculated into MRS liquid culture medium at an inoculum volume of 1% (V/V), cultured at 37° C. for 24 to 48 h, and then centrifuged at 6000× g for 10 min.

The bacterial cells were collected, rinsed twice with sterile physiological saline, and then resuspended in 1 mL of sterilized 0.1M KNO₃ solution to serve as the bacterial liquid to be tested.

2. Surface hydrophobicity determination: 50 μL of the above bacterial suspension was added into 2450 μL of 0.1M KNO₃, and the OD600 was recorded as A₀. 1.5 mL of the bacterial suspension was mixed with 500 μL of xylene and left at room temperature for 10 min (a two-phase system at this time). The two-phase system was vortexed for 2 min and left for 20 min to form the aqueous phase and the organic phase again. The aqueous phase was carefully removed (do not remove the organic phase) to measure the absorbance A₁ at 600 nm. Cell hydrophobicity was calculated according to the formula: Hydrophobicity %=(A₀−A₁)/A₁×%, and the average value was taken from three repeats.

The results show that the cell surface hydrophobicity of Lactobacillus reuteri VHProbi E18 provided by the present invention was 61.76%, and the standard deviation was 0.27%.

### Example 8 Cell adhesion test of Lactobacillus reuteri VHProbi E18

Caco-2 cells were thawed, subcultured, and amplified to the required amount. After adding trypsin, the cells were put back into the incubator. After observing with the naked eye that cells had completely detached and became as single cells as possible, the number of cells was counted using a hemocytometer, and PBS was used to dilute the cell suspension appropriately. The bacterial cells were then resuspended in MRS medium for later use. The bacterial suspension to be tested was incubated with Caco-2 cells for 2 h, and then non-adherent bacteria were washed away with PBS. After adding trypsin for digestion, cell culture medium was added to terminate digestion, and the liquid was collected for spread-plate counting. Adhesion capacity (CFU/cells)=total number of adherent bacteria in each culture well/total number of cells in each culture well.

After testing, Lactobacillus reuteri VHProbi E18 exhibited an adhesion capacity of 4.7.

### Example 9 Use of Lactobacillus reuteri VHProbi E18 in Improving the Skin of Aging Mice

### 1. Experimental Animals

C57 mice: SPF grade, male, six 3-month-old mice and eighteen 12-month-old mice, weighing 19-25 g. Environmental conditions for the feeding and management of experimental animals: room temperature 20-26° C., daily temperature difference ≤4° C., relative humidity 40-70%, and light and dark alternating time of 12/12 h. Animals were housed in standard mouse cages, with 6 animals per cage. Animal feed and drinking water: free access to food and water. The feed was SPF grade rat and mouse growth and breeding feed. Drinking water was high-temperature sterilized city tap water.

### 2. Experimental Methods

After 7 days of adaptive feeding, the mice were randomly divided into a 3-month-old control group, a 12-month-old control group, a 12-month-old smearing group, and a 12-month-old gavage group, with 6 mice in each group. The 12-month-old gavage group was given a probiotic liquid at 0.2 mL/10 g by gavage. The 12-month-old smearing group was applied the same amount of a probiotic liquid as the gavage group by smearing. The 3-month-old control group and the 12-month-old control group were given the same amount of normal saline as the probiotic liquid by gavage. The administration lasted for a total of 70 days.

### 3. Detection Indicators

At the end of the test, the sensory scores on the back fur of the mice were made, and the scoring rules are shown in Table 6. The changes in moisture content, SOD activity and MDA content of mouse skin were detected, and the hydroxyproline content in mouse skin and tail tendon was detected.

TABLE 6
Sensory score scale of mouse back fur

| Rating | Score | Appearance |
|---|---|---|
| 0 | 1 | Dull, dry, no oil color, split ends, |
| | | broken ends, white hair |
| + | 2 | Poor gloss, no oil color, white hair |
| ++ | 3 | Shiny, smooth hair, no white hair, black hair |
| +++ | 4 | Good luster, soft and shiny hair, glossy |
| | | and close-fitting fur, bright black |

After the test was completed, the back skin tissue was taken, fixed in 4% paraformaldehyde, extracted, dehydrated, embedded in paraffin, sectioned, and stained with HE to detect the number of hair follicles, dermal thickness, and sebaceous gland cells.

All experimental data are expressed as mean±standard deviation. Microsoft EXCEL was used for data statistics and graphing. The t test was used to compare the two groups of data, where P<0.05 was considered to be significantly different.

### 4. Experimental Results

### 4.1 Sensory Evaluation of Mouse Back Fur

At the end of the test, the hair of the 3-month-old mice was thick and neatly shiny; the mice in the 12-month-old control group had dry hair, more hair loss and gray hair; the mice in the 12-month-old smearing group had shiny hair and no white hair; and the mice in the 12-month-old gavage group had thick, shiny, and black hair. The comparison results of the sensory scores of the back fur of mice in each group are shown in FIG. 5.

### 4.2 Changes in Moisture Content, MDA and Hydroxyproline Content of Mouse Skin

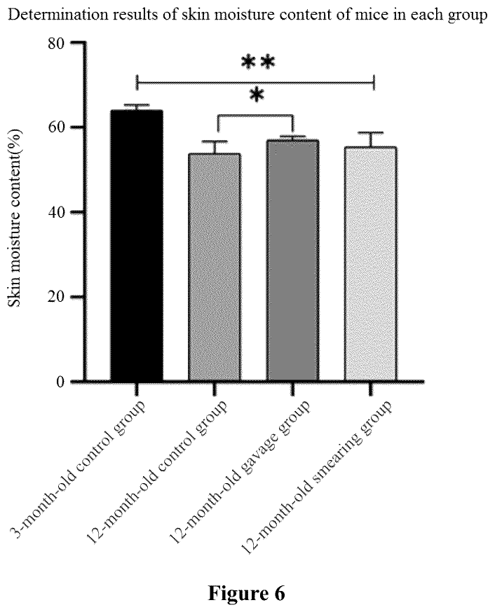

Compared with the 3-month-old control group at about 5-6 months of age, the skin moisture content of the 12-month-old groups was significantly lower (p<0.05), and the decrease rate was about 15.95%. The skin moisture content of the 12-month-old gavage group increased compared with the 12-month-old control group, with a significant difference (p<0.05). The comparison of skin moisture content of mice in each group is shown in FIG. 6.

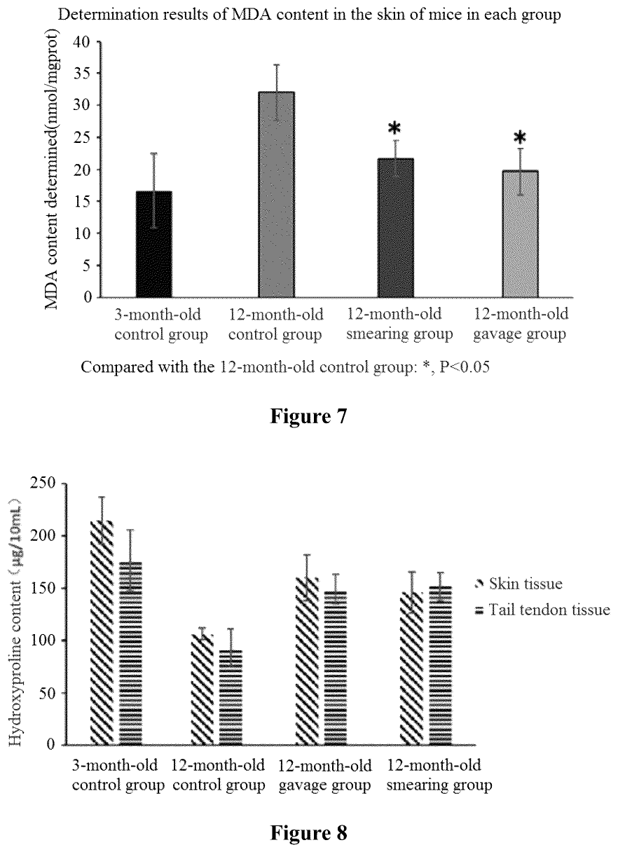

Compared with the 12-month-old control group, the MDA content in the skin of the 12-month-old gavage group was significantly reduced, with a significant difference (p<0.05). The MDA content in the skin of the 12-month-old smearing group was significantly reduced, also with a significant difference (p<0.05). There was no significant difference in the end-state skin MDA content between the 12-month-old smearing group and the 12-month-old gavage group (p>0.05). The comparison of the MDA content in the skin of each group is shown in FIG. 7.

Compared with the 12-month-old control group, the hydroxyproline content in the skin and tail tendons of the 12-month-old gavage group and the smearing group increased, with a significant difference (p<0.05). There was no significant difference in the end-state skin hydroxyproline content between the 12-month-old smearing group and the 12-month-old gavage group (p>0.05). The comparison of hydroxyproline content in the skin and tail tendons of mice in each group is shown in FIG. 8.

### 4.3 Histological Examination of Mouse Back Skin

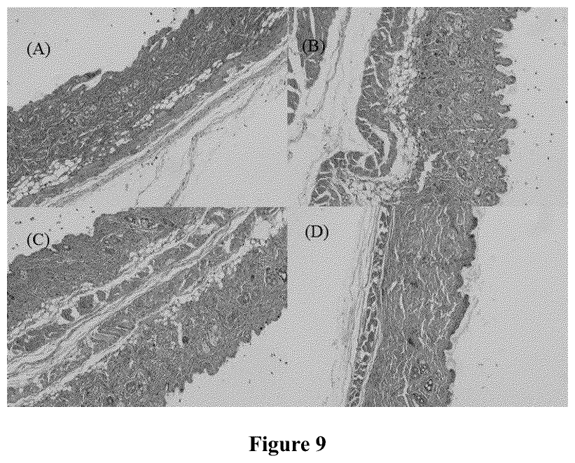

It can be seen under an optical microscope that the epidermal structure of the mice in the 3-month-old control group was intact, with clear cell layering, obvious epidermal layer and dermal papillae. No inflammatory cell infiltration was seen in the field of view. Sebaceous gland hyperplasia was visible, and dermal collagen fibers were visible in the field of view, arranged and distributed evenly into bands. The mice in the 12-month-old control group had significantly thinner epidermis, exfoliated stratum corneum, lack of structural integrity, reduced cell number, irregular arrangement, significantly reduced collagen fiber layer, broken, unevenly distributed and sparse fibers, and reduced fibroblasts. The mice in the 12-month-old gavage group had proliferated epithelial cells, with occasionally loose and disordered arrangements, sometimes hyperplastic hair follicles and sebaceous glands, occasionally inflammatory cells, and loosely distributed and visible broken collagen fibers. The mice in the 12-month-old smearing group had significantly proliferated epithelial cells, exfoliated stratum corneum, obviously hyperplastic hair follicles and sebaceous glands, visible inflammatory cells, loose skin, loosely arranged collagen fiber layer with obvious breakage. Typical HE section results of mouse skin are shown in FIG. 9.

At the end of the test, compared with the 3-month-old control group, the density of skin hair follicles of the mice in the 12-month-old control group mice decreased, with a significant difference (P<0.05); compared with the 12-month-old control group mice, the density of hair follicles of the mice in the 12-month-old smearing group and the 12-month-old gavage group increased, with a significant difference (P<0.05).

Compared with the 3-month-old control group, the skin epidermal thickness of the mice in the 12-month-old control group decreased, but with no significant difference; and there was no difference in the skin epidermal thickness of the mice in each 12-month-old group.

Compared with the 3-month-old control group, the skin dermis thickness of the mice in the 12-month-old control group decreased, with a significant difference (P<0.05); compared with the mice in the 12-month-old control group, the skin dermis thickness of the mice in the 12-month-old smearing group and in the 12-month-old gavage group increased, with a significant difference (P<0.05).

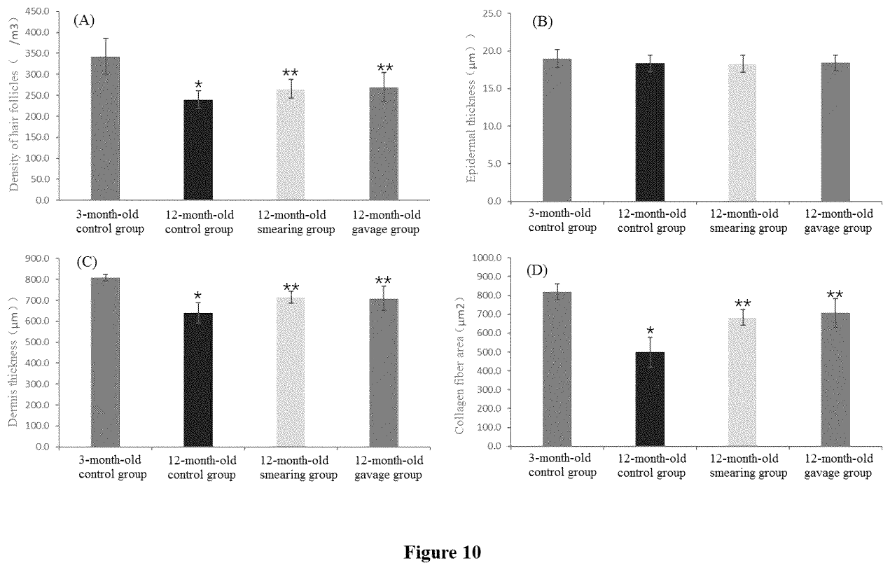

Compared with the 3-month-old control group, the skin collagen fiber area of the mice in the 12-month-old control group decreased, with a significant difference (P<0.05); compared with the 12-month-old control group, the skin collagen fiber area of the mice in the 12-month-old smearing group and in the 12-month-old gavage group increased, with a significant difference (P<0.05). The comparison results of the skin aging degree of mice in each group are shown in FIG. 10.

From the above results, it can be seen that compared with the mice in the 12-month-old control group, the glossiness of the fur of the mice in the 12-month-old gavage group and the 12-month-old smearing group was improved, with no white hair and less hair loss. For the mice in the 12-month-old gavage group and the 12-month-old smearing group, the skin moisture content increased, and the MDA content decreased. Hydroxyproline is an abundant and stable amino acid in the dermis. Its content can directly reflect the changes in collagen content in the dermis and is one of the indicators for detecting skin aging. The content of hydroxyproline in the skin and tail tendons of the mice in the 12-month-old gavage group and the 12-month-old smearing group increased, indicating that smearing and gavage of the Lactobacillus reuteri

VHProbi E18 strain can increase the content of collagen in the dermis. According to the histological examination of mouse skin, compared with the mice in the 12-month-old control group, the mice in the 12-month-old gavage group and the 12-month-old smearing group had an improved integrity of the epidermal structure, reduced inflammatory cells, and increased fibroblasts, and the effect of the gavage group was better than that of the smearing group. In addition, the number of skin hair follicles, dermis thickness, and collagen fiber content of the mice in the 12-month-old gavage group and 12-month-old smearing group increased. The results show that the Lactobacillus reuteri VHProbi E18 strain can improve the skin signs of aging mice and achieve the effect of delaying and repairing skin aging, whether administered by gavage or smearing.

In summary, the Lactobacillus reuteri VHProbi E18 provided by the present invention has strong tolerance to simulated artificial gastrointestinal fluid, which lays the foundation for the probiotic strain to successfully colonize in the colon through the gastrointestinal tract and exert its probiotic function. The antibiotic resistance test confirmed that Lactobacillus reuteri VHProbi E18 is sensitive to common antibiotics, does not produce hemolysin, and has good biological safety. Moreover, the Lactobacillus reuteri VHProbi E18 can scavenge DPPH and HRS, inhibit lipid peroxidation with certain antioxidant activity, can degrade cholesterol, and has prebiotic properties to reduce serum cholesterol. Animal experiments have demonstrated that Lactobacillus reuteri VHProbi E18 has anti-aging effects, can increase skin collagen content, improve hair follicle health, and enhance skin health. The Lactobacillus reuteri VHProbi E18 strain can be used to prepare food supplements or cosmetics having effects of delaying and repairing skin aging, and has broad application prospects.

1-8. (canceled)

9. A method for delaying skin aging, comprising administering a strain of Lactobacillus reuteri to a subject in need thereof.

10. The method according to claim 9, wherein the strain of Lactobacillus reuteri has been deposited in the China Center for Type Culture Collection on Jan. 25, 2021, with the deposit number of CCTCC NO: M2021153.

11. The method according to claim 9, wherein the strain of Lactobacillus reuteri has a fingerprint pattern as shown in FIG. 2.

12. The method according to claim 9, wherein the strain of Lactobacillus reuteri has a fingerprint pattern of RAPD as shown in FIG. 3.

13. The method according to claim 9, wherein the strain of Lactobacillus reuteri has a fingerprint pattern of rep-PCR as shown in FIG. 4.

14. The method according to claim 9, wherein the strain of Lactobacillus reuteri has a 16s rDNA sequence as shown in SEQ ID NO: 1.

15. The method according to claim 9, wherein the strain of Lactobacillus reuteri is administered orally or by smearing on the surface of skin.

16. The method according to claim 9, wherein the strain of Lactobacillus reuteri is in a form of food supplement.

17. A method for antioxidation, comprising administering a strain of Lactobacillus reuteri to a subject in need thereof.

18. The method according to claim 17, wherein the strain of Lactobacillus reuteri has been deposited in the China Center for Type Culture Collection on Jan. 25, 2021, with the deposit number of CCTCC NO: M2021153.

19. The method according to claim 17, wherein the strain of Lactobacillus reuteri has a fingerprint pattern as shown in FIG. 2.

20. The method according to claim 17, wherein the strain of Lactobacillus reuteri has a fingerprint pattern of RAPD as shown in FIG. 3.

21. The method according to claim 17, wherein the strain of Lactobacillus reuteri has a fingerprint pattern of rep-PCR as shown in FIG. 4.

22. The method according to claim 17, wherein the strain of Lactobacillus reuteri has a 16s rDNA sequence as shown in SEQ ID NO: 1.

23. The method according to claim 17, wherein the strain of Lactobacillus reuteri is administered orally or by smearing on the surface of skin.

24. The method according to claim 17, wherein the strain of Lactobacillus reuteri in a form of food supplement.


SEQUENCE LISTING (1 sequence)

SEQ ID NO: 1 - DNA, 1428 bp, Artificial Sequence
  Synthetic polynucleotide
aaaggttaggccaccgactttgggcgttacaaactcccatggtgtgacgggcggtgtgtacaaggcccgggaacgtattcaccgcggcatgctgatccgcgattactagcgattccgacttcgtgtaggcgagttgcagcctacagtccgaactgagaacggctttaagagattagcttactctcgcgagtttgcgactcgttgtaccgtccattgtagcacgtgtgtagcccaggtcataaggggcatgatgatctgacgtcgtccccaccttcctccggtttgtcaccggcagtctcactagagtgcccaactcaatgctggcaactagtaacaagggttgcgctcgttgcgggacttaacccaacatctcacgacacgagctgacgacgaccatgcaccacctgtcattgcgtccccgaagggaacgccttatctctaaggttagcgcaagatgtcaagacctggtaaggttcttcgcgtagcttcgaattaaaccacatgctccaccgcttgtgcgggcccccgtcaattcctttgagtttcaaccttgcggtcgtactccccaggcggagtgcttaatgcgttagctccggcactgaagggcggaaaccctccaacacctagcactcatcgtttacggcatggactaccagggtatctaatcctgttcgctacccatgctttcgagcctcagcgtcagttgcagaccagacagccgccttcgccactggtgttcttccatatatctacgcattccaccgctacacatggagttccactgtcctcttctgcactcaagtcgcccggtttccgatgcacttcttcggttaagccgaaggctttcacatcagacctaagcaaccgcctgcgctcgctttacgcccaataaatccggataacgcttgccacctacgtattaccgcggctgctggcacgtagttagccgtgactttctggttggataccgtcactgcgtgaacagttactctcacgcacgttcttctccaacaacagagctttacgagccgaaacccttcttcactcacgcggtgttgctccatcaggcttgcgcccattgtggaagattccctactgctgcctcccgtaggagtatggaccgtgtctcagttccattgtggccgatcagtctctcaactcggctatgcatcatcgccttggtaagccgttaccttaccaactagctaatgcaccgcaggtccatcccagagtgatagccaaagccatctttcaaacaaaagccatgcggcttttgttgttatgcggtattagcatctgtttccaaatgttatcccccgctccggggcaggttacctacgtgttactcacccgtccgccactcactggtgatccatcgtcaatcaggtgcaagcaccatcaatcagttgggccagtgcgtacgactgc